(19) United States
(12) Patent Application Publication
(10) Pub. No.: US 2022/0040052 A1
(43) Pub. Date: Feb. 10, 2022

(54) Compositions and Methods for Preparation of Solidified Oil for Treating Hair and Scalp

(71) Applicant: Tamiya Davis, Portsmouth, VA (US)

(72) Inventor: Tamiya Davis, Portsmouth, VA (US)

(73) Assignee: Tamiya Davis, Portsmouth, CA (US)

(21) Appl. No.: 17/394,332

(22) Filed: Aug. 4, 2021

Related U.S. Application Data
(60) Provisional application No. 63/061,154, filed on Aug. 4, 2020.

(51) Int. Cl. A61K 8/02; A61K 8/67; A61K 8/92; A61K 8/25; A61K 8/9789; A61Q 5/12; A45D 40/06

(52) U.S. Cl. CPC A61K 8/0229; A61K 8/678; A61K 8/922; A61K 8/25; A61K 8/9789; A61Q 5/12; A45D 2040/207; A61K 2800/48; A61K 2800/522; A61K 2800/524; A61K 2800/805; A61K 2800/87; A45D 40/06

(57) ABSTRACT

The present disclosure discloses a composition for solidified oil embodied in a dispenser for treating hair and scalp by moisturization. The composition comprising a blend of natural oils; essential oils; vitamins to provide antioxidants to the scalp; at least one thickening agent is mixed to increase the viscosity of the mixture formed; a tubular dispenser for holding the solidified oil composition formed from the mixture; a turning screw present on the bottom of the tubular dispenser for allowing the solidified oil composition to protrude out of the tubular dispenser when the turning screw is moved to an anti-clockwise direction, wherein the solidified oil composition is moved downwards when the turning screw is moved to a clockwise direction; and a cap for enclosing the tubular dispenser housed with the solidified oil composition.

### CROSS-REFERENCE TO RELATED APPLICATION

This disclosure claims the benefit of the priority of U.S. Provisional Patent Application No. 63/061,154, entitled “Scalp and Hair Oil Treatment Stick” and filed on Aug. 4, 2020. The above-identified application is incorporated herein by reference in its entirety.

### BACKGROUND

### Field of Invention

The present invention relates to hair and scalp moisturizing and hydrating compositions and methods of use. More particularly, the invention relates to a solidified oil composition and methods for promoting a healthy scalp and hair growth.

### Description of Related Art

Oiling the scalp is an ancient methodology grooming technique that has been practiced as a beauty regime among various ethnicities. Proper hair growth depends upon keeping the scalp in optimal condition by keeping the scalp conditioned, nourished, and moisturized. Although scalp skin contains sebaceous glands that secret sebum, a lubricating oil, to protect the skin from becoming too dry, the application of shampoos, chemical services, hair straighteners, cold weather, and hard water strips away sebum. Less sebum produced in the scalp causes dry scalp, poor hair health, and hair loss.

Carrier oils are used as scalp products to address this issue by emulating the natural sebum produced on the scalp. Essential oils and herbal plants are added to oil treatments to nourish scalp skin and provide healthy antioxidants and anti-inflammatory compounds. It is believed that these additional compounds help with hair growth by stimulating the scalp and increasing blood circulation that reduces scalp-related problems and dry hair.

Current products for oiling the scalp are typically provided in liquid form in either dropper bottles or applicator bottles and are poured directly onto the scalp. The oil usually spreads down the user's face, neck, ears, and clothing, creating an unpleasant condition. The oil also travels down the hair shaft causing the hair to become extremely oily and limp. The oil treatment must be washed out with shampoo and therefore defeat the purpose of adding moisture to the scalp.

Hair pomades and hair grease are thickened scalp moisturizers that are used as alternatives. However, they contain synthetic fragrances, mineral oil, and petroleum that create a thick heavy film on the scalp that causes oil stains on pillowcases, headrests, and oily skin. Synthetic fragrances added to scalp treatments may cause scalp irritation and can contain toxic chemicals. There is a strong need for a scalp oil treatment that is more convenient for use and addresses scalp issues mentioned previously.

### SUMMARY

Embodiments in accordance with the present invention provide a solidified oil embodied in a dispenser for treating hair and scalp by moisturization. The composition includes a blend of emollients, botanicals, and vitamins to nourish the scalp. The composition also includes at least one thickening agent to increase the viscosity of the mixture. Further, a tubular dispenser holds the solidified oil composition formed from the mixture. The tubular dispenser includes a turning screw present on the bottom of the tubular dispenser to allow the solidified oil composition to protrude out of the tubular dispenser when the screw is moved in a clockwise direction. The solidified oil composition is moved downward when the screw is moved in a counterclockwise direction. Further, the tubular dispenser includes a cap for enclosing the tubular dispenser housed with the solidified oil composition.

Embodiments in accordance with the present invention provide a method of preparing a solidified oil composition to have a solid oily wax feel for treating hair and scalp by moisturization. The method comprises steps of weighing the ingredients of the composition comprising a mixture of natural oils, waxes, natural plant butters, essential oils, minerals, and vitamins. The methods further include the step of heating the thickening agent comprising 12%-33% by weight of candelilla wax up to a temperature of 73° C. and allowing it to cool until 69° C. The method further includes the step of adding the oils, minerals, and butter into the thickening agent. The method further includes the step of adding essential oils and tocopherol at a temperature of 68.5° C. The method also includes the step of transferring the mixture to a mold dispenser and cooling the mixture to form a solidified oil composition having a solid oily wax feel.

Embodiments in accordance with the present invention provide a method of preparation of a solidified oil composition having a gel feel for treating hair and scalp by moisturization. Gelatinization agents comprise 4%-10% by weight to solidify the oil into a gel. The method includes mixing all oils except for avocado oil with C12-15 Alkyl Benzoate and lecithin and mix well. The method further includes the step of heating the mixture to 60° C. and cooling it to 40° C. The method further includes the step of mixing the extracts into the oil mixture. The method also includes the step of dissolving thickening agents selected from dibutyl lauroyl glutamide and dibutyl ethylhexanoyl glutamide in avocado oil at a temperature of 110° C. and cooling to a temperature of 80° C. The method also includes the step of pouring the gelling mixture slowly into the oil and extract mixture while constantly stirring. The method also includes the step of pouring the mixture immediately into mold dispensers to form the solidified oil composition. The method also includes the step of allowing the mixture to cool completely to obtain a solidified oil composition having an oily gel feel.

Further, the solidified oil composition is applied to the scalp before shampooing, after shampooing, and as needed. The solidified oil composition does not have to be rinsed or shampooed out. In use, the hair is parted in at least 2-inch sections, and the solidified oil composition is rubbed on the scalp. The consistency of the solidified oil composition will be light in texture and will melt on skin contact. Users are recommended to massage the oil into the scalp for 2 or 3 minutes.

Furthermore, hair oil users may more conveniently apply a scalp oil treatment via the solidified oil composition. The present invention will provide a skin scalp oil composition that remains on the scalp without causing the hair to become oily, limp, or drip down the user's body or unto clothing.

These and other advantages will be apparent from the present application of the embodiments described herein.

The preceding is a simplified summary to provide an understanding of some embodiments of the present invention. This summary is neither an extensive nor exhaustive overview of the present invention and its various embodiments. The summary presents selected concepts of the embodiments of the present invention in a simplified form as an introduction to the more detailed description presented below. As will be appreciated, other embodiments of the present invention are possible utilizing, alone or in combination, one or more of the features set forth above or described in detail below.

### BRIEF DESCRIPTION OF THE DRAWINGS

The above and still further features and advantages of embodiments of the present invention will become apparent upon consideration of the following detailed description of embodiments thereof, especially when taken in conjunction with the accompanying drawings, and wherein:

FIG. 1 illustrates a front view of a solidified oil composition embodied in a tubular dispenser with a turning screw for treating hair and scalp by moisturization, according to embodiments of the present invention disclosed herein;

FIG. 2 illustrates a schematic representation of the solidified oil composition embodied in a tubular dispenser with push-up means for treating hair and scalp by moisturization, according to embodiments of the present invention disclosed herein;

FIG. 3 illustrates a flowchart of a method for preparation of solidified oil composition having a solid oily wax feel for treating hair and scalp by moisturization, according to embodiments of the present invention disclosed herein; and

FIG. 4 illustrates a flowchart of a method preparation of a composition for solidified oil having gel feel for treating hair and scalp by moisturization, according to embodiments of the present invention disclosed herein.

The headings used herein are for organizational purposes only and are not meant to be used to limit the scope of the description or the claims. As used throughout this application, the word “may” be used in a permissive sense (i.e., meaning having the potential to), rather than the mandatory sense (i.e., meaning must). Similarly, the words “include”, “including”, and “includes” mean including but not limited to. To facilitate understanding, like reference numerals have been used, where possible, to designate like elements common to the figures. Optional portions of the figures may be illustrated using dashed or dotted lines unless the context of usage indicates otherwise.

### DETAILED DESCRIPTION

The following description includes the preferred best mode of one embodiment of the present invention. It will be clear from this description of the invention that the invention is not limited to these illustrated embodiments, but the invention also includes a variety of modifications and embodiments thereto. Therefore, the present description should be seen as illustrative and not limiting. While the invention is susceptible to various modifications and alternative constructions, it should be understood, that there is no intention to limit the invention to the specific form disclosed, but, on the contrary, the invention is to cover all modifications, alternative constructions, and equivalents falling within the spirit and scope of the invention as defined in the claims.

In any embodiment described herein, the open-ended terms “comprising,” “comprises,” and the like (which are synonymous with “including,” “having” and “characterized by”) may be replaced by the respective partially closed phrases “consisting essentially of,” consists essentially of,” and the like or the respective closed phrases “consisting of,” “consists of”, the like.

As used herein, the singular forms “a”, “an”, and “the” designate both the singular and the plural, unless expressly stated to designate the singular only.

The present invention provides a solidified oil for scalp treatment that promotes a healthy scalp by moisturization. The treatment and the product may be applied, before shampooing, to protect the scalp from harsh surfactants that cause dry scalp, between shampooing or as needed.

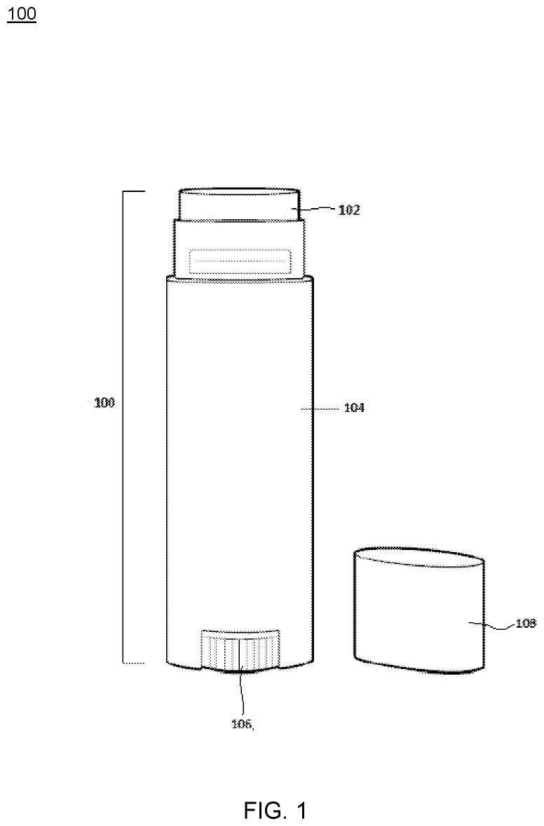

FIG. 1 illustrates a solidified oil composition embodied in a tubular dispenser with a turning screw 100 for treating hair and scalp by moisturization, according to embodiments of the present invention. The solidified oil composition embodied in a tubular dispenser with a turning screw 100 comprises a solidified composition 102, a tubular dispenser 104, a turning screw 106, and a cap 108, according to embodiments of the present invention.

Further, the solidified oil composition may be elliptical in shape with a length of 8.2 cm. In an embodiment of the present invention, the solidified oil composition 100 may comprise a diameter of 2.4 cm.

According to embodiments of the present invention, the solidified oil composition 102 may comprise a blend of natural oils. In an embodiment of the present invention, the natural oils may comprise 20%-50% by weight of jojoba oil, 15%-50% by weight of almond oil, 10-20% by weight evening primrose oil, 5%-10% by weight of black castor oil, and 5%-10% wheat germ oil by weight. Further, the natural oils may comprise 20%-50% by weight avocado oil, 10%-15% by weight sesame oil, 10%-20% by weight apricot oil, and 10%-20% by weight argan oil.

According to embodiments of the present invention, the solidified oil composition 102 may comprise essential oils. The essentials oils may stimulate the scalp for hair growth and have a soothing effect. According to an embodiment of the present invention, the essential oils may be selected from group comprises lavender oil, tea tree oil, rosemary oil, ylang-ylang oil, cedarwood oil, orange oil, peppermint oil, thyme oil, eucalyptus oil, neem oil, cinnamon leaf oil, clary sage oil, and lemongrass oil. Embodiments of the present invention are intended to include or otherwise cover any type of the essential oils including known, related art, and/or later developed technologies. Moreover, the essentials oils used may be in the range of 0.5%-5%.

According to embodiments of the present invention, the solidified oil composition 102 may comprise vitamins to provide antioxidants to the scalp. Further, the vitamins may be up to 1% by weight of tocopherol.

In an embodiment of the present invention, at least one thickening agent may be mixed to increase the viscosity of the mixture. According to embodiments of the present invention, the thickening agent may be selected from natural plant butters, waxes, and gelling agents either alone or in combination. Further, the plant butter may comprise 12%-25% by weight of shea butter, and wax may comprise 12%-33% by weight of candelilla wax. In an embodiment of the present invention, the thickening agent may be a gelling agent that may comprise 4%-10% of dibutyl lauroyl glutamide and dibutyl ethylhexanoyl glutamide relative to the total weight of the composition.

According to embodiments of the present invention, the solidified oil composition 102 may comprise 1%-2% by weight of silica. Moreover, the solidified oil composition 102 further comprises plant-derived extracts to relieve dryness, the extracts are selected from the group comprises hibiscus flower, aloe vera, ginger root extract, rose extract, licorice extract, bamboo extract, green tea extract, camelina extract, fenugreek extract, nettle extract, gooseberry extract, and alfalfa extract. In preferred embodiments of the present invention, the extracts may comprise 0.5%-10%. Herbal powders can be used in place of botanical extracts. Further, the solidified oil composition 102 may comprise 2%-15% by weight C12-15 Alkyl Benzoate as an emollient and texture enhancer, and 0.5%-5% by weight lecithin as an emulsifier.

Further, the solidified oil composition 102 may be housed in a tubular dispenser 104 enclosed with a cap 108. The tubular dispenser 104 may hold the solidified oil composition 102 formed from the mixture. The tubular dispenser 104 may comprise the turning screw 106 present on the bottom of the tubular dispenser 104 for allowing the solidified oil composition 102 to protrude out of the tubular dispenser 104 when the turning screw 106 is moved to a clockwise direction. The solidified oil composition 102 may be moved downwards when the turning screw 106 is moved to a counterclockwise direction. Furthermore, a tip of the solidified oil composition 102 may be smaller to align with measurements of hair parts for scalp application, and the solidified oil composition 102 may comprise compounds providing antioxidants, anti-inflammatory, and antimicrobials properties.

According to an embodiment of the present invention, the solidified oil composition 102 may be utilized for hydrating, nourishing, conditioning, and restoring the pH acid mantle of the scalp. In one embodiment of the present invention, the solidified oil composition 102 may contain ingredients selected from a group comprising, natural oils, carrier oils, essential oils, extracts, minerals, thickening agents, and so forth. Embodiments of the present invention are intended to include or otherwise cover any type of the ingredients including known, related art, and/or later developed technologies.

In an embodiment of the present invention, the thickening agents may be selected from a group comprising, waxes, gelling agents, natural plant butters, and so forth. Embodiments of the present invention are intended to include or otherwise cover any type of the thickening agents including known, related art, and/or later developed technologies. The thickening agents may be useful in solidifying the ingredients of the solidified oil composition 102. Further, the solidified oil composition 102 may be molded into stick form having a wax base. In another embodiment of the present invention, the solidified oil composition 102 may be molded into a stick form having a gel base.

In an embodiment of the present invention, the solidified oil composition 102 may be tubular in shape. In one embodiment, the solidified oil composition 102 may be composed of oils, plants, vitamins, and minerals mixed with the thickening agents.

In a preferred embodiment of the present invention, the solidified oil composition 102 oil may comprise 24% by weight jojoba oil, 18% by weight almond oil, 25% by weight candelilla wax, 12% by weight Shea butter, 8% by weight black castor oil, 6% by weight wheat germ oil, 2% by weight silica, 1% by weight tocopherol, 2% peppermint oil by weight, 2% rosemary oil by weight, and so forth.

Further, candelilla wax and Shea butter ingredients may form a solidified oil composition 102 while also providing moisturization. The solidifying ingredients may be used in combination or separately to solidify the mixture to obtain the solidified oil composition 102. According to embodiments of the present invention, the candelilla wax may be warmed to the maximum temperature of 73° C. Once cooled to 69° C., all the ingredients may be added except for the essential oils. When the mixture cools to 68.5° C. essential oils are added. The mixture is then transferred to mold dispensers to cool completely and form the solidified oil composition 102 with a solid oily wax feel.

According to the preferred embodiments of the present invention, the solidified oil composition 102 may comprise 30% by weight avocado oil, 15% by weight sesame oil, 12.5% by weight apricot oil, 9% by weight evening primrose oil, 10% by weight argan oil, 3% by weight dibutyl lauroyl glutamide, 3% dibutyl ethylhexanoyl glutamide, 5% by weight hibiscus extract, 5% by weight stinging nettle extract, 4% by weight C12-15 Alkyl Benzoate, 1% by weight thyme oil, 1% by weight lavender oil, 1% by weight Tocopherol, 0.5% by weight lecithin, and so forth.

Further, the ingredients of the solidified oil composition 102 used in the example above are typical ingredients used to moisturize the scalp. C12-15 Alkyl Benzoate and lecithin are premixed, and all ingredients are added to the mixture, except for the avocado oil. The mixture is constantly stirred and heated to about 60° C. and cooled to 40° C. Then the extracts are mixed into the oil mixture. The gelling agents dibutyl lauroyl glutamide and dibutyl ethylhexanoyl glutamide are then added to avocado oil at a temperature of 110° C. and cooled to a temperature of 80° C. The glutamide mixture is then slowly poured into the mixture while constantly stirring. Furthermore, the mixture may be poured immediately into mold dispensers to form a solidified oil with a solid gel feel.

According to embodiments of the present invention, the tubular dispenser 104 may hold the solidified oil composition 102 in a vertical position when housed inside the tubular dispenser 104.

According to embodiments of the present invention, the cap 108 may be configured for enclosing the tubular dispenser 104 housed with the solidified oil composition 102. In an exemplary scenario, a method for applying the solidified oil composition 102 to the skin is to part the hair and rub the solidified oil composition 102 ejected from the tubular dispenser 106 on the scalp by turning in a clockwise direction or pushing the material up as needed.

Further, when the application is completed, a cap 108 may be placed on the tubular dispenser 106 to enclose the tubular dispenser 104 housed with the solidified oil composition 102. According to embodiments of the present invention, the cap 108 may be made of a material such as, but not limited to, a plastic, a metal, a paper, and so forth. Embodiments of the present invention are intended to include or otherwise cover any type of material for cap 108 including known, related art, and/or later developed technologies.

The solidified oil composition 102 of the present invention may include non-limiting natural oils that provide penetrating compounds that may prevent dry scalp and protect the scalp from harsh ingredients found in hair care products.

In an embodiment of the present invention, the extracts may be poured in a mixture of the ingredients of the solidified oil composition 102 at 40° C. and Citric acid may be added to achieve a pH in the range of 3.5-5.5. Further, composition 102 may comprise compounds providing antioxidants, anti-inflammatory, antimicrobials properties.

According to embodiments of the present invention, the carrier oils may comprise oils, such as but not limited to, jojoba oil, meadowfoam oil, sweet almond oil, argan oil, olive oil, sunflower oil, safflower oil, sesame oil, avocado oil, coconut oil, grapeseed oil, black castor oil, wheat germ oil, palm oil, apricot kernel oil, evening primrose oil, and so forth.

According to embodiments of the present invention, the moisturizers may comprise esters, ceramides, fatty acids, and so forth. Embodiments of the present invention are intended to include or otherwise cover any type of the carrier oils including known, related art, and/or later developed technologies.

According to embodiments of the present invention, the vitamins used to reduce free radicals that cause hair follicles to break down and provide natural antioxidants may be, such as, but not limited to, vitamin E, biotin, pantothenic acid, provitamin B5/d-panthenol, vitaminB6 and so forth. Embodiments of the present invention are intended to include or otherwise cover any type of the Vitamins including known, related art, and/or later developed technologies.

According to embodiments of the present invention, the minerals such as phosphorous and silica (horsetail or Equisetum) increases hair growth and prevent excessive hair shedding/hair loss. Embodiments of the present invention are intended to include or otherwise cover any type of the minerals including known, related art, and/or later developed technologies. The preservatives may be selected from a group comprising, a citric acid, a tea tree oil, a glycerin, and so forth. Embodiments of the present invention are intended to include or otherwise cover any type of the preservatives including known, related art, and/or later developed technologies.

According to embodiments of the present invention, the plant butters may be used as the thickening agents that may comprise, babassu butter, monoi butter, kuku butter, mango butter, cocoa butter, Shea butter, karate butter, and so forth. Embodiments of the present invention are intended to include or otherwise cover any type of the carrier oils including known, related art, and/or later developed technologies. Embodiments of the present invention are intended to include or otherwise cover any type of natural plant butters including known, related art, and/or later developed technologies.

The plant butters may be used to boost viscosity and consistency because they are solid at room temperature. Other thickening agents may be used alongside or to replace the plant butters. These embodiments will solidify the ingredients to prevent the oil from dripping onto the user's clothing, face, and body when oiling the scalp.

Embodiments of the present invention are intended to include or otherwise cover any type of the essential oils including known, related art, and/or later developed technologies. The essential oils and the extracts may be used to stimulate hair growth by soothing the scalp, providing antimicrobial activity, and increasing blood circulation to the scalp. Further, citric acid may be used as a pH adjuster and a preservative.

According to embodiments of the present invention, the salts such as magnesium sulfate Epsom salt, sodium chloride (salt) are utilized to help with dandruff, excess oil absorption, and create volume. Embodiments of the present invention are intended to include or otherwise cover any type of the salts including known, related art, and/or later developed technologies.

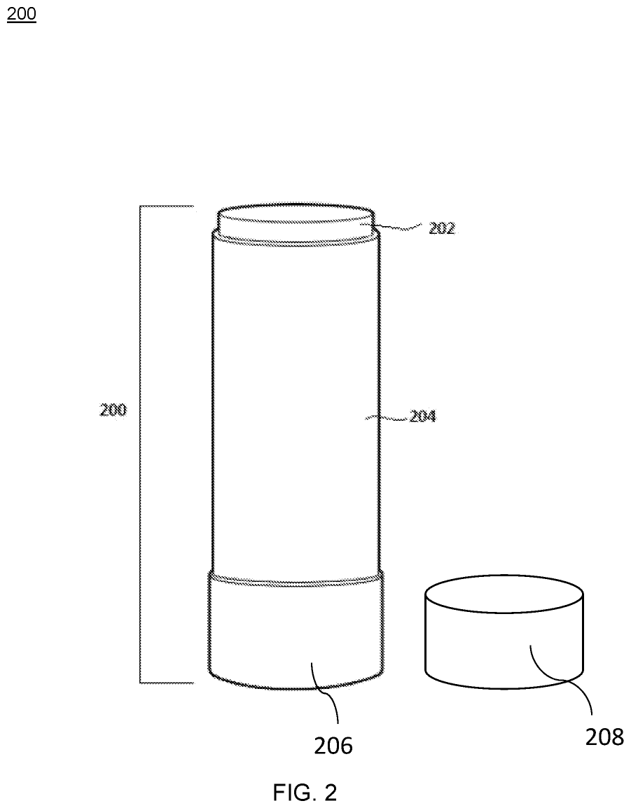

FIG. 2 illustrates a schematic representation of the solidified oil composition embodied in a tubular dispenser with push-up means 200 instead of a turning screw 106 as provided in FIG. 1 for treating hair and scalp by moisturization, according to embodiments of the present invention. The solidified oil composition embodied in a tubular dispenser with push-up means 200 comprises a solidified oil composition 202, a tubular dispenser 204, push-up means 206, and a cap 208, according to embodiments of the present invention.

Further, the solidified oil composition 202 may be housed in the tubular dispenser 204 enclosed with the cap 208. Furthermore, the beeswax, the mango butter, and the Shea butter may be added together and heated slowly to melt to form a mixture at 60° C., the remaining carrier oils may be added when the temperature is reached, and the mixture is cooled to 40° C. with the addition of essential oils and extracts.

According to embodiments of the present invention, the push-up means 206 may be present on the bottom of the tubular dispenser 204 for pushing the composition 202 up from the elliptical tubular dispenser 204 when required. In other embodiments of the present invention, the push-up means 206 may be present on the bottom of the tubular dispenser 204 for pushing the composition 202 up from the cylindrical tubular dispenser 204 when required. The tubular dispenser 204 may be a cylindrical tubular dispenser, an elliptical tubular dispenser, and so forth.

According to embodiments of the present invention, the cap 208 may be associated with the tubular dispenser 204 housed with the solidified oil composition 202 for the enclosure.

According to embodiments of the present invention, oils excluding avocado oil are weighed and added to the C12-15 Alkyl Benzoate and lecithin to form a mixture heated to about 72° C. and cooled to below 40° C., further the extracts are mixed in the oil mixture. Further, Dibutyl lauroyl glutamide and dibutyl hexanol glutamide are the gelling agents that form the oil into the stick, and the glutamides are dissolved in the avocado oil at a temperature of 110° C. and cooled to a temperature of 80° C. Then mixed into the oil and extract mixture with constant stirring and poured into mold dispensers.

According to embodiments of the present invention, the extracts may be selected from a group comprising, a hibiscus flower extract, an aloe vera leaf extract, a ginger root extract, a rice bran extract, a licorice extract, a bamboo extract, a green tea extract, a camelina extract, lemon peel extract, orange extract, lime extract, a fenugreek extract, a nettle extract, a gooseberry extract, and alfalfa extract, and so forth

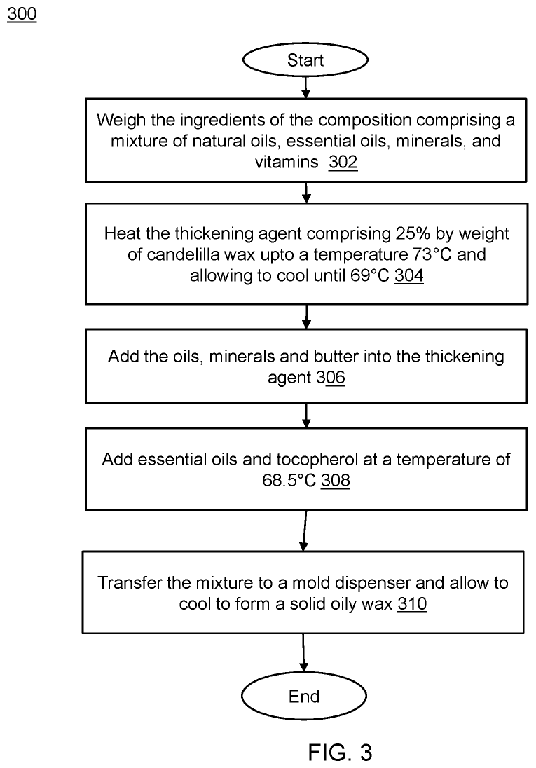

FIG. 3 illustrates a flowchart of a preparation of solidified oil composition having a solid oily wax feel for treating hair and scalp by moisturization, according to embodiments of the present invention.

At step 302, the ingredients of composition 102 comprising a mixture of natural oils, essential oils, minerals, and vitamins may be weighed. According to embodiments of the present invention, the ingredients of the composition 102 may comprise 20%-50% jojoba oil, 15%-50% almond oil, 12-25% shea butter, 5-10% black castor oil, 5-10% wheat germ oil, 1-2% silica, 1% tocopherol, 0.5-5% essential oil.

At step 304, the thickening agent may be comprising 12-33% by weight of candelilla wax may be heated up to a temperature of 73° C. and allowing cooling until a temperature around 69° C.

At step 306, the oils, minerals, and butter may be added to the thickening agent.

At step 308, the essential oils and tocopherol may be added at a temperature of 40° C.

At step 310, the mixture may be transferred to a mold dispenser and allowed to cool to form a solidified oil composition having a solid oily wax feel.

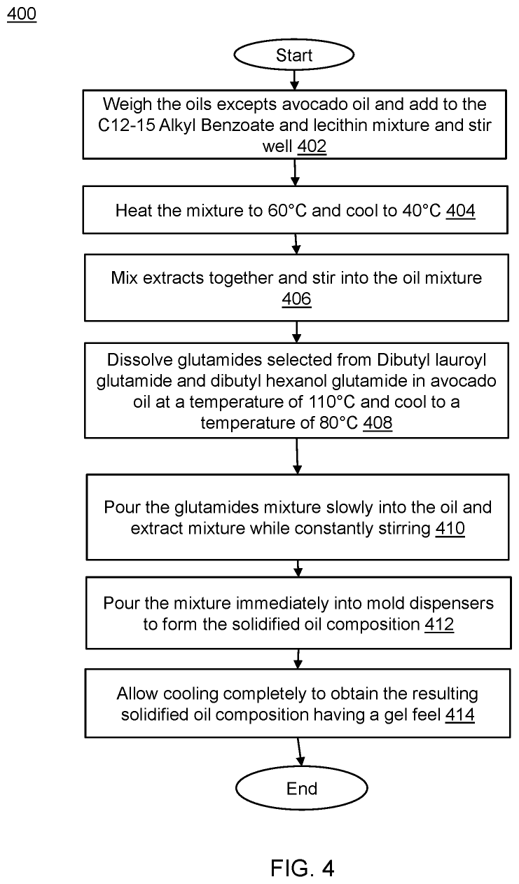

FIG. 4 illustrates a flowchart of a method for preparation of a composition for solidified oil having gel feel for treating hair and scalp by moisturization, according to embodiments of the present invention.

At step 402, the oils may be weighed except avocado oil and added to the C12-15 Alkyl Benzoate and lecithin mixture and mixed well.

At step 404, the mixture may be heated to 60° C. and cooled to 40° C.

At step 406, the extracts are mixed z may be mixed and then stirred into the oil mixture.

At step 408, glutamides selected from Dibutyl lauroyl glutamide and dibutyl hexanol glutamide may be dissolved in avocado oil at a temperature of 110° C. and cooled to a temperature of 80° C.

At step 410, the glutamides mixture is poured slowly into the oil and extract mixture while constantly being stirred.

At step 412, the mixture is poured immediately into mold dispensers to form the solidified oil composition.

At step 414, allow cooling completely to obtain the resulting solidified oil composition having gel feel.

Further, the composition may comprise natural oils comprise 20-50% avocado oil, 10-15% sesame oil, 10-20% apricot oil, 10-20% argan oil, gelling agents comprise 4-10% dibutyl lauroyl glutamide and dibutyl hexanol glutamide, plant derived extracts comprise 0.5-5% hibiscus extract, 0.5-5% stinging nettle extract, natural wax comprises 12%-33% candelilla wax, 2-15% C12-15 Alkyl Benzoate, 0.5-5% essential oil, 1% Tocopherol, and 0.5-5% lecithin.

While the invention has been described in connection with what is presently considered to be the most practical and various embodiments, it is to be understood that the invention is not to be limited to the disclosed embodiments, but on the contrary, is intended to cover various modifications and equivalent arrangements included within the spirit and scope of the appended claims.

This written description uses examples to disclose the invention, including the best mode, and also to enable any person skilled in the art to practice the invention, including making and using any devices or systems and performing any incorporated methods. The patentable scope of the invention is defined in the claims and may include other examples that occur to those skilled in the art. Such other examples are intended to be within the scope of the claims if they have structural elements that do not differ from the literal language of the claims, or if they include equivalent structural elements within substantial differences from the literal languages of the claims.

What is claimed is:

1. A composition for solidified oil embodied in a tubular dispenser for treating hair and scalp by moisturization, the composition comprising:
a blend of natural oils;
essential oils;
vitamins to provide antioxidants to the scalp;
at least one thickening agent mixed to increase the viscosity of the mixture formed,
wherein the tubular dispenser holds the solidified oil composition formed from the mixture includes,
a turning screw or a push-up means present on the bottom of the tubular dispenser for allowing the solidified oil composition to protrude out of the tubular dispenser; and
a cap for enclosing the tubular dispenser housed with the solidified oil composition.

2. The composition as claimed in claim 1, wherein the essentials oils stimulate the scalp for hair growth and have a soothing effect, the essential oils are selected from group comprises lavender oil, tea tree oil, rosemary oil, ylang-ylang oil, cedarwood oil, orange oil, peppermint oil, thyme oil, eucalyptus oil, neem oil, cinnamon leaf oil, clary sage oil, lemongrass oil, and/or a combination thereof.

3. The composition as claimed in claim 1, wherein the thickening agent are selected from natural plant butters, waxes, and gelling agents either alone or in combination.

4. The composition as claimed in claim 3, wherein the natural botanical butters are used as the thickening agents that comprise babassu butter, monoi butter, kuku butter, mango butter, cocoa butter, Shea butter, karate butter, and/or a combination thereof.

5. The composition as claimed in claim 3, wherein the natural plant butter comprises 12%-25% by weight of shea butter, and wax comprises 12%-33% by weight of candelilla wax.

6. The composition as claimed in claim 1, wherein the composition comprises 1%-2% by weight of silica.

7. The composition as claimed in claim 1, wherein a tip of the solidified oil composition is smaller to align with measurements of hair parts for scalp application and the solidified oil composition comprises compounds providing antioxidants, anti-inflammatory, antimicrobials properties.

8. The composition as claimed in claim 1, wherein the vitamins are 1% by weight of tocopherol.

9. The composition as claimed in claim 1, wherein the natural oils comprise 20%-50% by weight of avocado oil, 10%-15% by weight of sesame oil, 10%-20% by weight of apricot oil, and 10%-20% argan oil.

10. The composition as claimed in claim 1, wherein the thickening agent is a gelling agent comprises 4%-10% by weight of dibutyl lauroyl glutamide and dibutyl hexanol glutamide.

11. The composition as claimed in claim 1, wherein the composition further comprises 5%-10% by weight plant-derived extracts to relieve dryness, the extracts are selected from group comprises hibiscus flower, aloe vera, ginger root extract, rice bran extract, licorice extract, bamboo extract, green tea extract, camelina extract, fenugreek, nettle, gooseberry, and alfalfa.

12. The composition as claimed in claim 1, wherein the composition comprises 2%-15% by weight C12-15 Alkyl Benzoate and 0.5-5% by weight lecithin as an emulsifier.

13. The composition as claimed in claim 1, wherein the natural oils comprise 20-50% by weight of jojoba oil, 15-50% by weight of almond oil, 5-10% by weight of black castor oil, and 5-10% wheat germ oil.

14. The composition as claimed in claim 1, wherein the composition comprises preservatives are selected from a group comprising, a citric acid, a tea tree oil, a glycerin, and/or a combination thereof.

15. The composition as claimed in claim 1, wherein the push-up means present on the bottom of the tubular dispenser for pushing the solidified oil composition up from the tubular dispenser when required.

16. The composition as claimed in claim 1, wherein the turning screw is rotated to a clockwise direction to move the solidified oil composition up and turned an anti-clockwise direction to move the solidified oil composition downward.

17. A method for preparation of solidified oil composition having a solid oily wax feel for treating hair and scalp by moisturization, the method comprises steps of:
a. weighing the ingredients of the composition comprising a mixture of natural oils, essential oils, minerals, and vitamins;
b. heating the thickening agent comprising 25% by weight of candelilla wax up to a temperature 73° C. and allowing to cool until 69° C.;
c. adding the oils, minerals, and butter into the thickening agent;
d. adding essential oils and tocopherol at a temperature of 68.5° C.; and
e. transferring the mixture to a mold dispenser and allowing cooling to form solidified oil composition having a solid oily wax feel.

18. The method as claimed in claim 17, wherein the ingredients of the composition comprise 20%-50% jojoba oil, 15%-50% almond oil, 12-25% shea butter, 5-10% black castor oil, 5-10% wheat germ oil, 1-2% silica, 1% tocopherol, 0.5-5% essential oil.

19. A method for preparation of a composition for solidified oil having gel feel for treating hair and scalp by moisturization, the method comprises steps of:
a. weighing oils except for avocado oil and added to the C12-15 Alkyl Benzoate and lecithin and mixed well;
b. heating the mixture to 60° C. and cooling the mixture below 40° C.;
c. mixing the extracts and stirring into the oil mixture;
d. dissolving glutamides selected from Dibutyl lauroyl glutamide and dibutyl hexanol glutamide in avocado oil at a temperature of 110° C. and cooling to a temperature of 80° C.;
e. pouring the glutamides mixture slowly into the oil and extract mixture while constantly stirring;
f. pouring the mixture immediately into mold dispensers to form the solidified oil composition; and
g. allowing cooling completely to obtain the resulting solidified oil composition having gel feel.

20. The method as claimed in claim 19, wherein the composition comprises:
natural oils comprise 20-50% avocado oil, 10-15% sesame oil, 10-20% apricot oil, 10-20% argan oil, gelling agents comprise 4-10% dibutyl lauroyl glutamide and dibutyl hexanol glutamide, plant derived extracts comprise 0.5-5% hibiscus extract, 0.5-5% stinging nettle extract, natural wax comprises 2-15% C12-15 Alkyl Benzoate, 0.5-5% essential oil, 1% Tocopherol, and 0.5-5% lecithin.
